(19) United States
(12) Patent Application Publication
(10) Pub. No.: US 2013/0090524 A1
(43) Pub. Date: Apr. 11, 2013

(54) Self-inflating Dildo

(71) Applicant: Paul McNamara, Northampton, MA (US)

(72) Inventor: Paul McNamara, Northampton, MA (US)

(73) Assignee: Paul J. McNamara, Northampton, MA (US)

(21) Appl. No.: 13/200,973

(22) Filed: Oct. 6, 2011

(51) Int. Cl. A61F 5/00

(52) U.S. Cl. 600/38

(57) ABSTRACT

An inflatable form comprising a distal portion that is generally elongate and cylindrical in shape and a proximal portion that is generally bulbous, providing a grasping and manipulation means. The inflatable form is engaged with a comfortable surface, also known as a comfort-sleeve that is fitted over the elongate portion that is intended to be inserted into the body cavity. Inflatable forms provide a compact, discrete item that may be reused or may be disposed of after use.

### BACKGROUND

Among items available for enhancing sexual pleasure are dildos and prostate stimulators. These items are comprised of a variety of materials, including silicone, plastic, wood, etc. and are generally elongate and cylindrical in shape. Some are comprised of flexible bladders that are inflated with fluid such as liquid or air. Their length, width, and shape vary within anatomically prescribed limits. The distal end may have a handle means such as a bulbous portion for manipulating the object. In use they are inserted into the vagina or anus, either by an individual or by a partner, typically using suitable lubrication. They provide stimulation from friction upon manipulation of the object, or by pressure from the distension of the cavity due to the volume or in the case of inflatable embodiments, by the changing volume of the object. Some objects create diffuse sensations in the user, or they may have surface texture, such as raised bumps or ridges which enhance local pressures upon manipulation of the object.

### SUMMARY

Example embodiments described herein include, but are not limited to inflatable forms that are generally elongate and cylindrical in shape providing a grasping and manipulation means and a comfortable surface, also known as a comfort-sleeve over the portions that are intended to be inserted into the body cavity. Inflatable forms provide a compact, discrete item that may be reused or may be disposed of after use.

In an example embodiment, an inflatable form comprised of two elastic planes of a semi-flexible polymer such as mylar that is heat-sealed into a contiguous-closed container. Inflation is provided by at least one capsule filled with a chemical that reacts with elements in the container or with a chemical in a second capsule to form a gas that provides inflation of the form. Chemicals employed are typically non-toxic materials that are comprised of an alkaline component such as sodium bicarbonate, one or more acid salts, an inert starch and a liquid for means of hydration and activation. Baking soda is the source of the carbon dioxide, and the acid-base reaction can be generically represented as:

NaHCO₃+H⁺→Na⁺+CO₂+H₂O

### BRIEF DESCRIPTION OF THE DRAWINGS

A more complete understanding of the components, processes, and apparatuses disclosed herein may be obtained by reference to the accompanying figures. These figures are intended to illustrate the teachings taught herein and are not intended to show relative sizes and dimensions, or to limit the scope of examples or embodiments. In the drawings, the same numbers are used throughout the drawings to reference like features and components of like function.

FIG. 1 is a left front perspective view of an example embodiment of an inflated sexual stimulation device including an inflatable form and a comfort-sleeve

FIG. 2 is an exploded, perspective view of the inflatable portion of the embodiment of FIG. 1.

FIG. 3 is a front, orthographic view of the inflatable portion in the inflated state, of the device of FIG. 1.

FIG. 4 is a front, orthographic view of the device of FIG. 1.

FIG. 5 is a left front perspective, exploded view of the device of FIG. 1.

### DETAILED DESCRIPTION

Example embodiments described herein relate to an inflatable sexual stimulation device. In one embodiment of the present invention, an inflatable sexual stimulation device may include a mated pair of heat-sealed polymeric planes surrounding at least one capsule that when broken, allows two chemicals to combine. Heat-sealed polymeric material is a common method of creating inflatable products such as balloons. Although not as flexible as elastomeric materials, the heat-sealed polymers offer benefits including strength characteristics and printing capabilities. A covering comprised of flexible elastomeric material such as latex is fitted over the inflatable form.

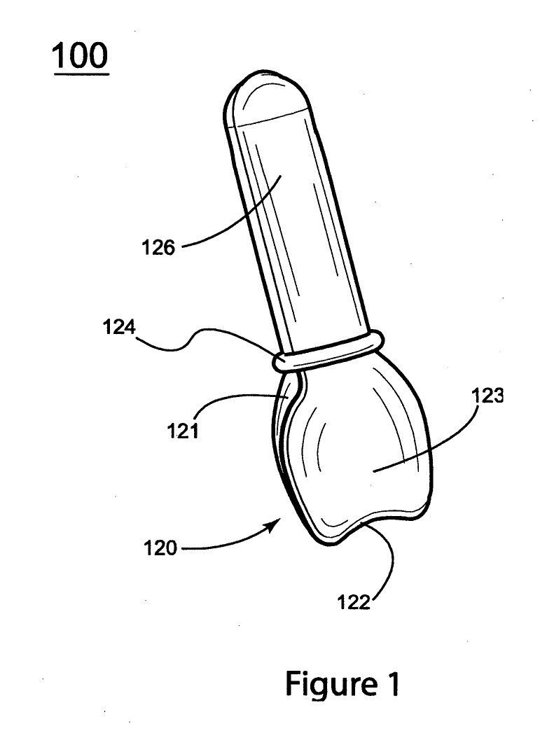

FIG. 1 depicts the sexual stimulation device in an inflated state comprising an inflatable form 120 that further comprises a front plane 123 and a rear plane 121 that is seamed together along a perimeter seam 122. The inflatable form generally comprises a bulbous handle portion and an elongated portion. A comfort-sleeve 126 is fitted over the elongated portion of the inflatable form. A ring 124 is comprised of elastomeric material such as neoprene or silicone and provides a physical, sensual reference to denote the end of the elongated portion of the inflatable sexual stimulation device.

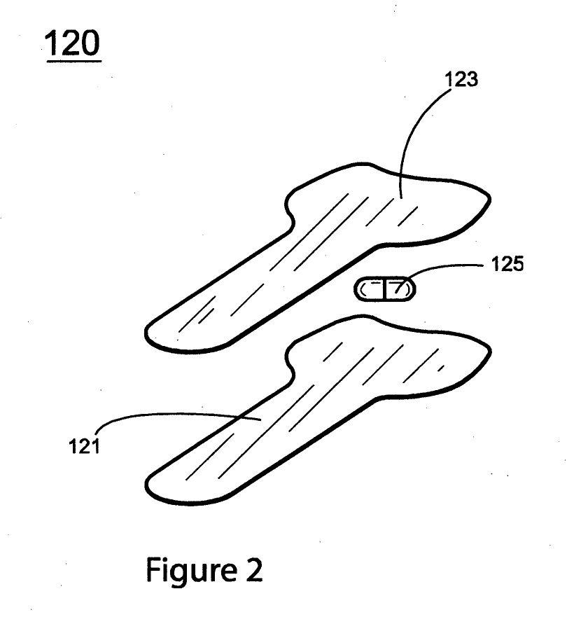

Referring to FIG. 2 an exploded view of the inflatable form is depicted. One side 123 (referred to here because of the drawing orientation, as the upper side) is a planar form that is cut from a sheet of polymeric material that can be heat-sealed, such as mylar. The lower side, 121 is as well, a planar form that is cut from a sheet of polymeric material that can be heat-sealed, such as mylar. Encased between the two sides is at least one capsule 125. In the embodiment shown in FIG. 2, the capsule has two compartments such that when it is broken, the material contained in the two compartments combines. These materials create a gas that is safe for internal use though not specifically intended as such.

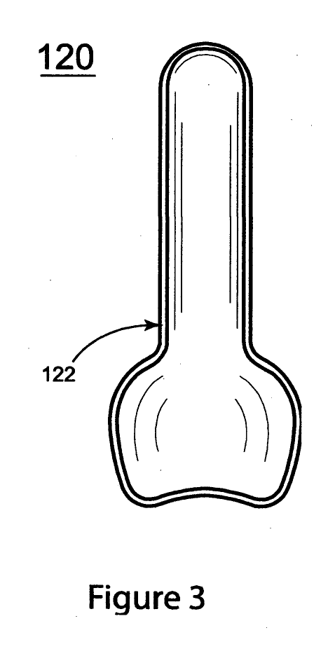

Referring to FIG. 3, an orthographic view of the sealed inflatable form of FIG. 2 is depicted. A seam 122 is the result of the heat-sealing process. Such a seam creates a piece of material with an edge that may be uncomfortable to sensitive skin.

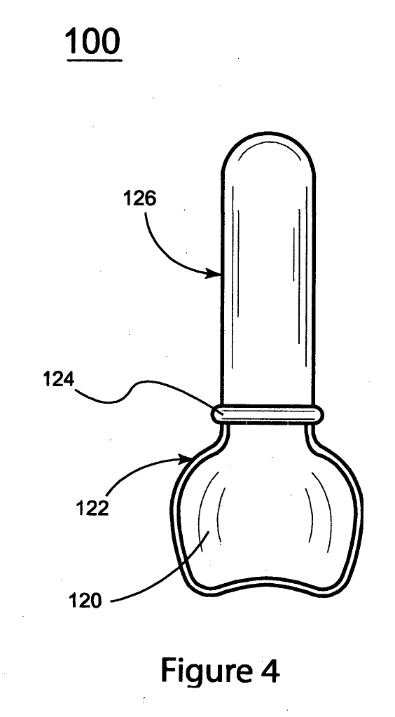

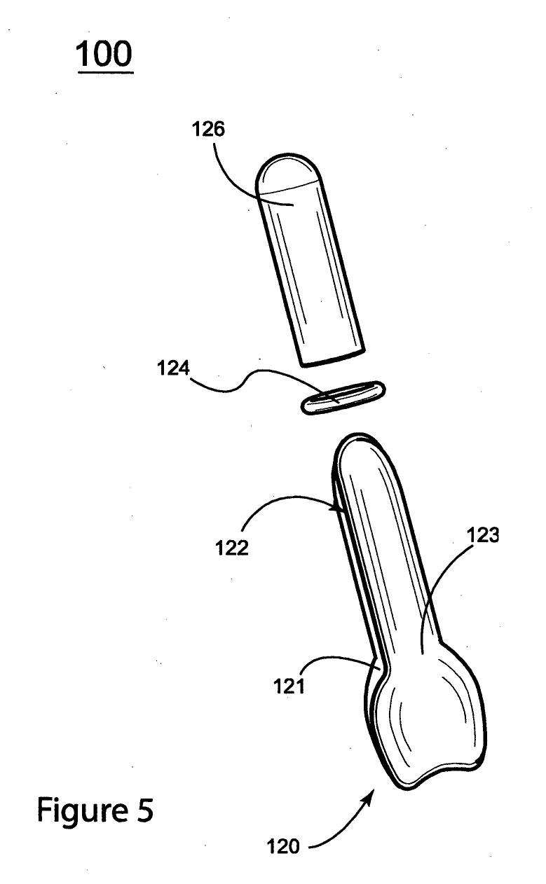

Referring to FIG. 4, and FIG. 5, FIG. 4 is an orthographic, front view and FIG. 5 is a left front exploded view of the heat-sealed inflated form comprising the comfort sleeve 126 and elastomeric ring 124. The inflatable form 120 provides a bulbous, proximal end and an elongated distal end. An elastomeric sleeve 126 is fitted over the elongated, distal end and provides a means of mitigating any uncomfortable sensation that would otherwise be felt by sensitive skin in contact with the seam 122. An elastomeric ring 124 is engaged with the elongated portion of the inflated form 120, such that one may feel the presence of the elastomeric ring 124 before inserting the embodiment further into the body cavity at which point contact with the seam 122 might occur. The bulbous, proximal end of the inflatable form 120 provides a gripping means for the embodiment.

1. A device for insertion into a vaginal canal comprising a:
an inflatable member containing a pneumatic source substantially housed within said inflatable member to inflate said inflatable member;
wherein said pneumatic source comprises components chemically reacting to produce a non-toxic gas, said components being contained within said inflatable member:
an elongated, generally cylindrical distal portion; and
a generally bulbous proximal portion; and

2. The device of claim 1, further comprising an elastomeric portion engaged with said elongated, generally cylindrical distal portion.

3. The device of claim 2 further comprising an elastomeric ring engaged with the region between said elongated portion and said bulbous portion.
